(19) United States
(12) Patent Application Publication
(10) Pub. No.: US 2026/0124022 A1
(43) Pub. Date: May 7, 2026

(54) Dental Appliance With Sensor Module

(71) Applicant: Otis Dental, Inc., Covina, CA (US)

(72) Inventor: Andrew Lee, San Francisco, CA (US)

(21) Appl. No.: 18/937,485

(22) Filed: Nov. 5, 2024

(51) Int. Cl. A61C 7/08; A63B 71/08

(52) U.S. Cl. CPC A61C 7/08; A63B 71/085; A61B 5/01; A61B 5/0816; A61B 5/087; A61B 5/4818; A61B 5/682; A61B 2562/0219; A61B 2562/0247; A61B 2562/0261

(57) ABSTRACT

In one embodiment, a dental appliance includes a dental-appliance body and a core sensor module embedded within the dental-appliance body. The core sensor module includes one or more processors. The dental appliance further includes at least one biological sensor coupled to the dental-appliance body and conductively coupled to the core sensor module, and further includes a conductive layer running through at least a portion of the dental-appliance body and conductively coupling the core sensor module with the at least one biological sensor.

### TECHNICAL FIELD

This application generally relates to a dental appliance having a sensor module.

### BACKGROUND

Many dental appliances are designed to be worn within a user's mouth. For instance, braces are orthodontic devices that fit over, or attach to, a user's teeth to straighten or move that user's teeth. Aligners serve a similar purpose, while retainers are often used to maintain teeth position, i.e., to prevent or lessen teeth movement over time. Mouthguards are typically worn over the teeth and can be used in variety of settings, including to prevent bruxism or to reduce force transfers to the head while playing sports. Dentures are false teeth that are used in place of a user's real teeth.

### BRIEF DESCRIPTION OF THE DRAWINGS

FIG. 1 illustrates an example of a mouthguard dental appliance.

FIG. 2 illustrates an example computing system.

FIG. 3 illustrates an embodiment of the example mouthguard dental appliance of FIG. 1.

### DESCRIPTION OF EXAMPLE EMBODIMENTS

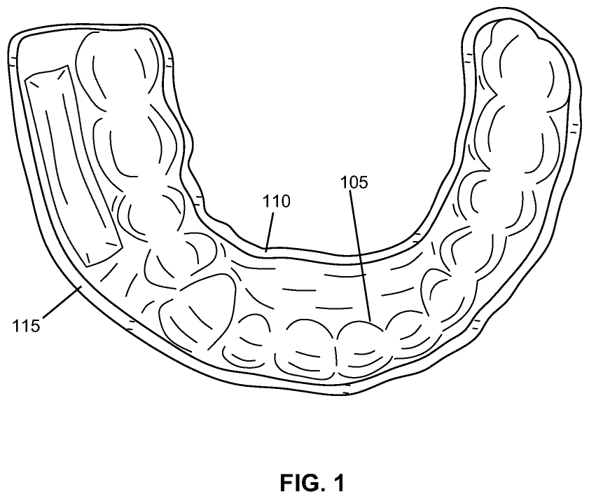

Many dental appliances are designed to be worn within a user's mouth, including dental appliances such as braces, aligners, mouthguards (including nightguards, sports guards, etc.), dentures, and so on. FIG. 1 illustrates an example of a mouthguard dental appliance designed to be worn on the upper teeth. The mouthguard in the example of FIG. 1 is molded from a polymer material, although mouthguards and other dental appliances made by made from other materials. In the example of FIG. 1, a user's teeth fit within interior portion 105 of the mouthguard, which is formed by back wall 110 and front wall 115, each of which has a particular thickness (which may be the same thickness or may be different thicknesses). Back wall 110 is positioned behind the user's teeth while being worn, and front wall 115 is positioned in front of the user's teeth.

A dental appliance, such as the example mouthguard of FIG. 1, may include a sensor module to which one or more sensors can be conductively coupled (electrically connected). For example, a dental appliance may include a core sensor module that includes one or more microprocessors and associated memory and may include a communication module (e.g., a Bluetooth module, a WiFi module, an NFC module, etc.), a power source such as a rechargeable battery, charging components for charging the power source, and basic sensors such as an accelerometer. Particular embodiments of the core sensor module may include more than these components or less than these components.

The core sensor module may be embedded on or within the dental appliance. For instance, in the example of FIG. 1, a core sensor module may be embedded within the mouthguard, for example in the right side (i.e., the portion of the mouthguard worn on the right side of the user's mouth) of the mouthguard. However, in general a core sensor module may be embedded in other regions of a dental appliance.

One or more biological sensors may couple to the core sensing module to provide the dental appliance with a range of biological sensing capabilities. For example, a PPG sensor may be used to detect heartrate, respiratory rate and other breathing-related metrics, blood oxygenation, saliva chemistry and pH, and so on. A dental appliance may obtain a good-quality signal from a PPG sensor, at least because the mouth is a relatively dark environment, and therefore spurious ambient light will not dilute the PPG's sensors readings. A microphone may detect breathing-related metrics, and may also be used to detect conditions such as sleep apnea (for instance, a microphone may be embedded in a night-worn mouthguard, retainers, or aligners, and the dental appliance may then be used to detect sleep apnea). Other sensors such as an accelerometer may be used a biological sensor, in particular embodiments.

A bite sensor may include a force sensor (which may include a pressure sensor) for a detecting a bite force of the user's teeth. The force sensor may be a capacitive sensor, a resistive sensor, or another suitable force-sensing modality. A bite sensor may be used to detect, e.g., bruxism of a wearer.

In particular embodiments, a dental appliance may include three biological sensors: (1) a brux sensor; (2) a breath sensor and (3) a salivary pH sensor. In particular embodiments, the brux sensor and the salivary sensor may be located on opposite sides of the dental appliance. For example, a brux sensor may be located on the right (or left) side of a dental appliance, while the salivary sensor may be located on the left (or right) side, respectively, of the appliance. The salivary sensor may be located at or near the outside of a front wall (e.g., front wall 115 in the example of FIG. 1) of the dental appliance. For instance, the salivary pH sensor may be located on the facial/buccal side of the dental appliance, near the Stensen salivary duct on one side of the user's mouth, thereby increasing the detection efficacy of the salivary sensory. The force sensor may then be located on the opposite of the dental appliance. In particular embodiments, a brux sensor and a salivary pH sensory may be approximately symmetrically balanced across the dental appliance; e.g., if the brux sensor is located near the molar teeth on the right side, then the salivary sensor may be located near the molar teeth on the left side. In particular embodiment, depending on a user's dental condition and on the dental appliance used, a bite sensor and/or a pH sensor may be moved forward from the molar teeth, e.g., if a user does not have premolar teeth. In some embodiments, a force sensor and a salivary pH sensor may be co-located in a dental appliance.

In particular embodiments, a breathing sensor may be located behind the user's teeth, for example near the two front center teeth. For instance, with reference to the example mouthguard of FIG. 1, a breath sensor may be located at or near the outside of back wall 110, near the center two teeth. As a result, breath exiting the wearer's throat will encounter the sensor at or near 180 degrees from the center of the sensor's field of view (e.g., breath movement and the sensor's sensing area are approximately parallel to each other, pointing in opposite directions). In other embodiments, the breath sensor may be moved to other portions of the dental appliance, but still may be located at or near the outside of the back of the dental appliance, facing the palatal/lingual portion of the user's mouth.

In particular embodiments, a dental appliance having a bite sensor, a breathing sensor, and a pH sensor may have those sensors approximately symmetrically balanced throughout the dental appliance. For instance, one sensor (e.g., a force sensor) may be located on one side of the appliance and toward the rear of the appliance, while another sensor (e.g., a salivary sensor) may be located on the other side of the appliance, toward the rear of the appliance. A breathing sensor may be located approximately equidistant from these two sensors, so that the three sensors are roughly symmetrically balanced in the appliance. However, this disclosure contemplates that other physical configurations may be used.

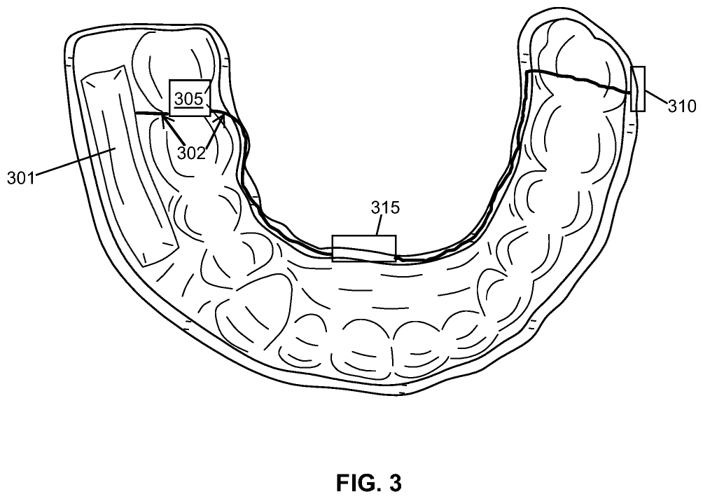

FIG. 3 illustrates an example embodiment of the example mouthguard dental appliance of FIG. 1. The example mouthguard of FIG. 3 includes a core sensor module 301. A force sensor 305 is located on one side of the example mouthguard of FIG. 3, and a salivary sensor 310 is located opposite the force sensor, on the other side of mouthguard. In the example of FIG. 3, salivary sensory is located on a front wall, cheek-facing portion of the mouthguard. The example mouthguard of FIG. 3 further includes a breathing sensor 315, which is located on a back wall, lingual facing portion of the mouthguard. The example of FIG. 3 illustrates an embodiment of conductive layer 302 coupling each biological sensor 305, 310, and 315 to core sensor module 301.

In particular embodiments, a dental appliance may include additional or other biological sensors. For example, a biological sensor may include an accelerometer or other suitable IMU sensor, which can be used to track a user's motion, track a user's sleep state, etc.

In particular embodiments, a core sensor module may include a printed circuit board that contains at least some of the core sensor module's circuitry. Each biological sensor may electrically connect to the core sensor module. For example, a flexible copper layer (e.g., encased in a polymer layer) may extend from the core senor module to each of the biological sensors. The copper layer may include a power bus for providing power to a biological sensor from the core sensor module's power source, and may include one or more conductive data lines for transmitting data between the sensor module and the biological sensor. The copper layer may take the form of a conductive ribbon that is embedded within the body of the dental appliance (e.g., within a polymer body of the example mouthguard of FIG. 1). Thus, in particular embodiments a single power source and core sensor module (e.g., CPU, memory, etc.) may supply electrical power and processing capabilities to a distributed set of biological sensors that are placed in separate locations throughout the body of the dental appliance.

The body of a dental appliance may be shaped as a plastic/polymer appliance for teeth such as, by way of example, a ready-made unfitted appliance, a mouth-adapted appliance adapted by heating and placing in mouth, a custom-made appliance made from a dental impression, an appliance made by 3D printing, etc. Components of the core sensing module and/or of the biological sensors may be miniaturized mechanical and/or electro-mechanical elements that are made and integrated together using techniques of microfabrication, and such components may be integrated into an encapsulation that is millimeters in size. In particular embodiments, a dental appliance may include communication components, such as an antenna, for wirelessly or wired transmission of information to another computing device, such as a smartphone or a personal computer.

In particular embodiments, a dental appliance may have an associated charging station, such as a case that includes charging components for charging a power supply of a core sensor module of the dental appliance. For example, a dental appliance's power supply may include an induction coil for charging the battery through an inductive coupling with another induction coil, such as from a wireless charging station, which may be part of case for the dental appliance. In particular embodiments, an antenna may include an induction coil that can be inductively coupled to charge the battery, and thus the power supply does not need a separate induction coil for charging. In particular embodiments, a dental appliance may include a passive power supply, i.e. may have no battery, in which case the sensing components may be intermittently activated when an antenna or a separate induction coil receives an external charge, e.g., from another computing device or an electrical outlet.

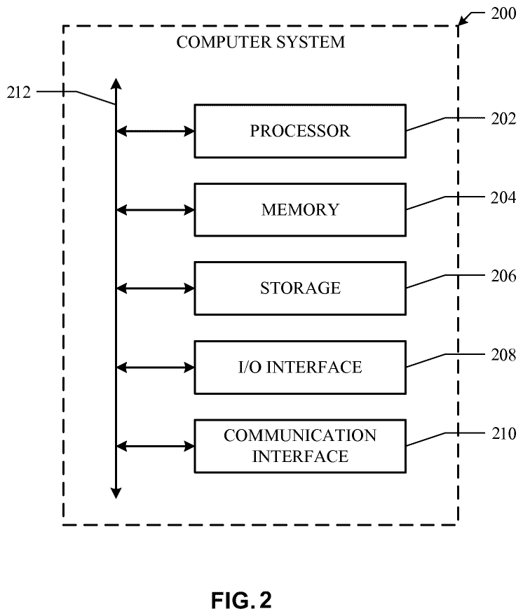

FIG. 2 illustrates an example computer system 200. In particular embodiments, one or more computer systems 200 perform one or more steps of one or more methods described or illustrated herein. In particular embodiments, one or more computer systems 200 provide functionality described or illustrated herein. In particular embodiments, software running on one or more computer systems 200 performs one or more steps of one or more methods described or illustrated herein or provides functionality described or illustrated herein. Particular embodiments include one or more portions of one or more computer systems 200. Herein, reference to a computer system may encompass a computing device, and vice versa, where appropriate. Moreover, reference to a computer system may encompass one or more computer systems, where appropriate.

This disclosure contemplates any suitable number of computer systems 200. This disclosure contemplates computer system 200 taking any suitable physical form. As example and not by way of limitation, computer system 200 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 200 may include one or more computer systems 200; be unitary or distributed; span multiple locations; span multiple machines; span multiple data centers; or reside in a cloud, which may include one or more cloud components in one or more networks. Where appropriate, one or more computer systems 200 may perform without substantial spatial or temporal limitation one or more steps of one or more methods described or illustrated herein. As an example and not by way of limitation, one or more computer systems 200 may perform in real time or in batch mode one or more steps of one or more methods described or illustrated herein. One or more computer systems 200 may perform at different times or at different locations one or more steps of one or more methods described or illustrated herein, where appropriate.

In particular embodiments, computer system 200 includes a processor 202, memory 204, storage 206, an input/output (I/O) interface 208, a communication interface 210, and a bus 212. Although this disclosure describes and illustrates a particular computer system having a particular number of particular components in a particular arrangement, this disclosure contemplates any suitable computer system having any suitable number of any suitable components in any suitable arrangement.

In particular embodiments, processor 202 includes hardware for executing instructions, such as those making up a computer program. As an example and not by way of limitation, to execute instructions, processor 202 may retrieve (or fetch) the instructions from an internal register, an internal cache, memory 204, or storage 206; decode and execute them; and then write one or more results to an internal register, an internal cache, memory 204, or storage 206. In particular embodiments, processor 202 may include one or more internal caches for data, instructions, or addresses. This disclosure contemplates processor 202 including any suitable number of any suitable internal caches, where appropriate. As an example and not by way of limitation, processor 202 may include one or more instruction caches, one or more data caches, and one or more translation lookaside buffers (TLBs). Instructions in the instruction caches may be copies of instructions in memory 204 or storage 206, and the instruction caches may speed up retrieval of those instructions by processor 202. Data in the data caches may be copies of data in memory 204 or storage 206 for instructions executing at processor 202 to operate on; the results of previous instructions executed at processor 202 for access by subsequent instructions executing at processor 202 or for writing to memory 204 or storage 206; or other suitable data. The data caches may speed up read or write operations by processor 202. The TLBs may speed up virtual-address translation for processor 202. In particular embodiments, processor 202 may include one or more internal registers for data, instructions, or addresses. This disclosure contemplates processor 202 including any suitable number of any suitable internal registers, where appropriate. Where appropriate, processor 202 may include one or more arithmetic logic units (ALUs); be a multi-core processor; or include one or more processors 202. Although this disclosure describes and illustrates a particular processor, this disclosure contemplates any suitable processor.

In particular embodiments, memory 204 includes main memory for storing instructions for processor 202 to execute or data for processor 202 to operate on. As an example and not by way of limitation, computer system 200 may load instructions from storage 206 or another source (such as, for example, another computer system 200) to memory 204. Processor 202 may then load the instructions from memory 204 to an internal register or internal cache. To execute the instructions, processor 202 may retrieve the instructions from the internal register or internal cache and decode them. During or after execution of the instructions, processor 202 may write one or more results (which may be intermediate or final results) to the internal register or internal cache. Processor 202 may then write one or more of those results to memory 204. In particular embodiments, processor 202 executes only instructions in one or more internal registers or internal caches or in memory 204 (as opposed to storage 206 or elsewhere) and operates only on data in one or more internal registers or internal caches or in memory 204 (as opposed to storage 206 or elsewhere). One or more memory buses (which may each include an address bus and a data bus) may couple processor 202 to memory 204. Bus 212 may include one or more memory buses, as described below. In particular embodiments, one or more memory management units (MMUs) reside between processor 202 and memory 204 and facilitate accesses to memory 204 requested by processor 202. In particular embodiments, memory 204 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 204 may include one or more memories 204, where appropriate. Although this disclosure describes and illustrates particular memory, this disclosure contemplates any suitable memory.

In particular embodiments, storage 206 includes mass storage for data or instructions. As an example and not by way of limitation, storage 206 may include a hard disk drive (HDD), a floppy disk drive, flash memory, an optical disc, a magneto-optical disc, magnetic tape, or a Universal Serial Bus (USB) drive or a combination of two or more of these. Storage 206 may include removable or non-removable (or fixed) media, where appropriate. Storage 206 may be internal or external to computer system 200, where appropriate. In particular embodiments, storage 206 is non-volatile, solid-state memory. In particular embodiments, storage 206 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 206 taking any suitable physical form. Storage 206 may include one or more storage control units facilitating communication between processor 202 and storage 206, where appropriate. Where appropriate, storage 206 may include one or more storages 206. Although this disclosure describes and illustrates particular storage, this disclosure contemplates any suitable storage.

In particular embodiments, I/O interface 208 includes hardware, software, or both, providing one or more interfaces for communication between computer system 200 and one or more I/O devices. Computer system 200 may include one or more of these I/O devices, where appropriate. One or more of these I/O devices may enable communication between a person and computer system 200. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 208 for them. Where appropriate, I/O interface 208 may include one or more device or software drivers enabling processor 202 to drive one or more of these I/O devices. I/O interface 208 may include one or more I/O interfaces 208, where appropriate. Although this disclosure describes and illustrates a particular I/O interface, this disclosure contemplates any suitable I/O interface.

In particular embodiments, communication interface 210 includes hardware, software, or both providing one or more interfaces for communication (such as, for example, packet-based communication) between computer system 200 and one or more other computer systems 200 or one or more networks. As an example and not by way of limitation, communication interface 210 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 210 for it. As an example and not by way of limitation, computer system 200 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 200 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 200 may include any suitable communication interface 210 for any of these networks, where appropriate. Communication interface 210 may include one or more communication interfaces 210, where appropriate. Although this disclosure describes and illustrates a particular communication interface, this disclosure contemplates any suitable communication interface.

In particular embodiments, bus 212 includes hardware, software, or both coupling components of computer system 200 to each other. As an example and not by way of limitation, bus 212 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 212 may include one or more buses 212, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend.

1. A dental appliance comprising:
a dental-appliance body;
a core sensor module embedded within the dental-appliance body, the core sensor module comprising one or more processors;
a plurality of biological sensors, each coupled to the dental-appliance body and conductively coupled to the core sensor module, comprising (1) a force sensor located on one side of the dental appliance (2) a salivary sensor located on a second side of the dental appliance opposite the one side on which the force sensor is located and (3) a breathing sensor located towards a middle of the dental appliance; and
a conductive layer running through at least a portion of the dental-appliance body and conductively coupling the core sensor module with each of the plurality of biological sensors.

2. (canceled)

3. The dental appliance of claim 1, wherein the force sensor comprises a capacitive sensor or a resistive sensor.

4. (canceled)

5. The dental appliance of claim 1, wherein the breathing sensor comprises one or more of (1) a PPG sensor or (2) a microphone.

6-8. (canceled)

9. The dental appliance of claim 1, wherein the force sensor and the salivary sensor are located toward a rear portion of the dental appliance.

10. The dental appliance of claim 1, wherein the salivary sensor is located on a front wall, cheek-facing portion of the dental appliance.

11. The dental appliance of claim 1, wherein the breathing sensor is located on a back wall, lingual-facing portion of the dental appliance.

12. The dental appliance of claim 1, wherein the dental appliance comprises a mouthguard.

13. The dental appliance of claim 1, wherein the core sensor module further comprises a power supply.

14. The dental appliance of claim 13, wherein the conductive layer couples the power supply of the core sensor module to each of the plurality of biological sensors.

15. The dental appliance of claim 1, wherein the core sensor module further comprises one or more wireless communication components.

16. The dental appliance of claim 1, further comprising an accelerometer coupled to the core sensor module.
